(19) United States
(12) Patent Application Publication
(10) Pub. No.: US 2018/0147364 A1
(43) Pub. Date: May 31, 2018

(54) Drug Delivery Device with Multifunctional Bias Structure

(71) Applicant: Novo Nordisk A/S, Bagsvaerd (DK)

(72) Inventors: Simon Roervig, Copenhagen OE (DK); Carsten Schau Andersen, Valby (DK)

(21) Appl. No.: 15/575,516

(22) PCT Filed: May 26, 2016
(86) PCT No.: PCT/EP2016/061907

§ 371 Date: Nov. 20, 2017

(30) Foreign Application Priority Data
May 28, 2015 (EP) 15169635.8

(51) Int. Cl. A61M 5/315; A61M 5/20

(52) U.S. Cl. CPC A61M 5/31581; A61M 5/31551; A61M 2005/202; A61M 2205/581; A61M 2205/582; A61M 5/2033

(57) ABSTRACT

The present invention provides a drug delivery device comprising a housing (2) extending along a longitudinal axis, a dose dial member (3) being rotatable relative to the housing (2) to set a dose of drug to be expelled from an attached reservoir and comprising a first serrated transversal surface (7), a second serrated transversal surface (16) being axially and rotationally fixed with respect to the housing (2), and being configured for slipping engagement with the first serrated transversal surface (7) in a bi-directional ratchet interface in response to the dose dial member (3) being rotated relative to the housing (2) to increase or decrease the dose, and a dose delivery mechanism for expelling a set dose, the dose delivery mechanism comprising a piston rod (50), a torsion spring (30) for providing energy to drive the piston rod (50), an activation button (8) being axially movable relative to the housing (2) between an inactivated position in which the torsion spring (30) is retained and an activated position in which the torsion spring (30) is released to cause an expelling of the set dose, the activation button (8) being biased towards the inactivated position by a bias structure (40) arranged to act between the dose dial member (3) and the activation button (8), the bias structure (40) thereby further biasing the first serrated transversal surface (7) towards the second serrated transversal surface (16), and a drive member (20) being rotationally biased by the torsion spring (30), wherein the drive member (20) is axially fixed to the activation button (8) and axially movable relative to the housing (2) between a dose setting position in which the drive member (20) is rotationally fixed to the dose dial member (3) and a dose expelling position in which the drive member (20) is rotationally freed from the dose dial member (3) and rotationally interlocked with the piston rod (50).

### FIELD OF THE INVENTION

The present invention relates generally to spring powered drug delivery devices, and more specifically to ratchet mechanisms for use in such devices.

### BACKGROUND OF THE INVENTION

Drug delivery devices, such as injection devices, are widely used for administration of liquid drugs to people in need of therapeutic treatment. Many injection devices are capable of repeated setting and injection of either a fixed or a variable volume of drug upon operation of respective dose setting and dose expelling mechanisms in the device. Some injection devices are adapted to be loaded with a prefilled drug reservoir containing a volume of drug which is sufficient to provide for a number of injectable doses. When the reservoir is empty, the user replaces it with a new one and the injection device can thus be used again and again. Other injection devices are prefilled when delivered to the user and can only be used until the drug reservoir has been emptied, after which the device is discarded. The various injection devices comprise a dose expelling mechanism which typically expels the drug by advancing a piston in the reservoir using a motion controlled piston rod.

Some injection devices require the user to depress a push button a certain distance towards a housing to thereby manually cause the piston rod to pressurise the reservoir and advance the piston therein for expelling of a dose. The force which must be applied to the push button to perform such an operation is often not insignificant and may cause handling problems for people with reduced finger strength and/or dexterity.

Automatic injection devices offering automatic expelling of a dose of drug in response to a release of a cocked spring are popular because the spring, once released, provides all the energy needed to complete the injection. Such devices typically only require the user to apply a small, short-duration force to trigger the injection. The spring can either be arranged to be strained before each injection, or it can be pre-strained, e.g. by the manufacturer, to store energy sufficient to occasion an emptying of the drug reservoir in one or more injections.

WO 2014/161952 (Novo Nordisk A/S) discloses an automatic injection device utilising a helical torsion spring to provide energy for advancement of a piston in a drug cartridge. The spring is strained by the user as part of the dose setting procedure. A ratchet mechanism comprising a radially deflectable arm is employed to prevent the spring from unwinding during the dose setting.

WO 2008/031235 (Tecpharma Licensing AG) discloses an alternative automatic injection device which also includes a torsion spring powered drive. In this device an axial ratchet mechanism holds the torque from the spring during dose setting. A dedicated ratchet ring is held axially displaceably in a rotatable dosing sleeve and is biased elastically by a plurality of springs against an axially fixed toothed surface, establishing a slip coupling. This solution requires not only an additional component in the form of a ratchet ring but also several dedicated spring elements to enable the slip coupling effect. These additional components all add to the total number of parts in the injection device and thereby to the manufacturing costs thereof.

WO 2014/166908 (Sanofi) discloses a torsion spring driven injection device where an injection button return spring is employed to elastically support an axial ratchet mechanism serving to hold the torque from the spring during dose setting. This solution appears to eliminate the need for dedicated spring elements to enable the slip coupling effect. However, a dedicated return spring component is still required in addition to the injection button return spring to automatically place the injection device in the dose setting mode following an injection.

So, while an axial ratchet mechanism may be preferable in some designs the prior art solutions are not optimised in view of the general desires to reduce the amount of waste from, and the manufacturing costs of, drug delivery devices, desires which are particularly pronounced in the area of disposable drug delivery devices, where the entire product is discarded after it's emptied of the drug substance.

### SUMMARY OF THE INVENTION

It is an object of the invention to eliminate or reduce at least one drawback of the prior art, or to provide a useful alternative to prior art solutions.

In particular, it is an object of the invention to provide a low cost spring powered drug delivery device which offers simple and reliable dose setting and dose adjustment.

It is a further object of the invention to provide a spring powered drug delivery device having a mode switching mechanism which is composed of a reduced number of components.

It is an even further object of the invention to provide a spring powered drug delivery device having low constructional complexity.

In the disclosure of the present invention, aspects and embodiments will be described which will address one or more of the above objects and/or which will address objects apparent from the following text.

Thus, in one aspect the invention provides a drug delivery device according to claim 1.

Thereby, a drug delivery device, such as e.g. an injection device, may be provided comprising a housing, e.g. at least generally cylindrical, extending along a longitudinal axis, means for receiving a drug reservoir, a dose dial member rotatable relative to the housing to set a dose of drug to be expelled from a received reservoir, a first serrated transversal surface being rotationally fixed with respect to the dose dial member, a second serrated transversal surface being axially and rotationally fixed with respect to the housing, and being configured for slipping engagement with the first serrated transversal surface, and a dose delivery mechanism for expelling a set dose.

The dose delivery mechanism comprises a piston rod being distally displaceable relative to the housing, a drive spring in the form of a torsion spring, and an activation button, or injection button, being axially movable relative to the housing from an inactivated position to an activated position to release the drive spring and execute the expelling of the set dose. The dose dial member is rotationally biased by the drive spring during dose setting, and at least a portion of the dose dial member is configured to move axially relative to the housing between an engaged position, in which the first serrated transversal surface and the second serrated transversal surface are fully engaged, and an overhaul position, in which respective apices of the first serrated transversal surface are about to pass respective apices of the second serrated transversal surface, in response to the dose dial member being rotated relative to the housing to increase or decrease the dose.

The drug delivery device further comprises a bias structure arranged to act between the dose dial member and the activation button, biasing the dose dial member towards the engaged position and the activation button towards the inactivated position, and a drive member for conveying energy from the drive spring to the piston rod, e.g. via one or more intermediate elements. The drive member is axially fixed with respect to the activation button and thus axially movable, in response to an axial movement of the activation button, between a dose setting position, corresponding to the activation button being in the inactivated position, and a dose expelling position, corresponding to the activation button being in the activated position. The drive member is furthermore rotationally fixed with respect to the dose dial member in the dose setting position and rotationally released from the dose dial member in the dose expelling position, and the position of the drive member thus defines the particular mode of the drug delivery device in that when the drive member is in the dose setting position the drug delivery device is in a dose setting mode, i.e. a state wherein a dose may be set, and when the drive member is in the dose expelling position the drug delivery device is in a dose delivery mode, i.e. a state wherein the drive spring is released to cause a pressurisation of the received reservoir.

The first serrated transversal surface, the second serrated transversal surface, and the bias structure thus together constitute an axial ratchet mechanism suitable for holding the dose dial member in a plurality of possible angular positions relative to the housing against the torque of the drive spring, thereby resisting unwinding of the drive spring during dose setting. Each of the plurality of possible angular positions of the dose dial member relative to the housing may correspond to a unique dose size to be expelled from the drug delivery device.

The above defined solution leverages on the widespread implementation in spring powered drug delivery devices of an injection button return mechanism in that it utilises the presence of an injection button biasing element to provide the required elastic support for the ratchet coupling between the first serrated transversal surface and the second serrated transversal surface as well as the energy for returning the drive member to the dose setting position following a dose administration, automatically placing the drug delivery device in the dose setting mode. Thereby, no dedicated bias components need be added to the construction, saving additional expenses in that respect.

The drive member may be axially fixed to the activation button, respectively rotationally fixed to the dose dial member in the dose setting position, either directly or via one or more intermediate elements. Similarly, the drive member may be configured to interlock with the piston rod in, or during movement to, the dose expelling position directly or via one or more intermediate elements. For example, the drive member may be configured to rotationally interlock with a rotatable piston rod guide being configured to engage and rotate the piston rod through a nut member in the housing for expelling of the set dose. The interlocking of the drive member with the piston rod may take place prior to, or at the latest simultaneously with, the rotational release of the drive member from the dose dial member.

The activation button may be arranged at a proximal end portion of the housing, in which case the inactivated position may be a position in which at least an operable portion of the activation button extends proximally from the proximal end portion of the housing, and the activated position may be a position in which less of the activation button extends from the proximal end portion of the housing than in the inactivated position, or even in which the activation button is fully depressed in the housing. Alternatively, the activation button may be slidably arranged at a side portion of the housing.

The bias structure may comprise any suitable structure capable of exerting an elastic force on the respective components, such as e.g. a compression spring, an air spring, a foam rubber, etc. In any case the bias structure is chosen such that the force it provides is sufficient for the first serrated transversal surface and the second serrated transversal surface to hold the torque from the drive spring also when the largest possible dose is set.

In particular, the bias structure may be a unitary element comprising a distal end portion arranged in abutment with a proximally oriented portion, such as e.g. an interior flange portion, of the dose dial member, and a proximal end portion arranged in abutment with a distally oriented portion, such as e.g. an underside portion, of the activation button.

The bias structure may be arranged concentrically about the longitudinal axis to provide a symmetrical force distribution to both the dose dial member and the activation button, thereby i.a. minimising any circumferential pressure variation between the first serrated transversal surface and the second serrated transversal surface.

The bias structure may have a temperature independent, or substantially temperature independent, force characteristic in the range [−10° C.; 50° C.] to ensure a reliable dose setting mechanism under normal use conditions. In particular, the bias structure may be or comprise a metallic compression spring. Further, the bias structure may be made of a nonmagnetic material, e.g. to avoid influencing a magnetic field. In some embodiments, the bias structure is or comprises a plastic spring.

In particular embodiments of the invention the drive member and the drive spring are arranged concentrically with the housing and with the piston rod, thereby providing a slender pen-like configuration of the drug delivery device. One exemplary version of such a drug delivery device is a pen injection device, which is further characterised by having a cartridge type reservoir (comprising a penetrable septum and an axially slidable piston) arranged in longitudinal extension of the dose delivery mechanism. Pen injection devices are widely used within diabetes care for delivery of various glucose regulating agents, such as e.g. insulin or glp-1. These injection devices are either pre-loaded with a drug cartridge by the manufacturer during production or configured to receive a drug cartridge provided by a user. In some exemplary manifestations of the present invention the drug delivery device is a pen injection device of the former type, and in other exemplary manifestations of the present invention the drug delivery device is a pen injection device of the latter type.

The first serrated transversal surface may form part of the dose dial member. This ensures that any rotation of the dose dial member is accompanied by an identical rotation of the first serrated transversal surface and further eliminates the need for an additional dedicated ratchet component in the device. In particular, the first serrated transversal surface may be formed, e.g. circumferentially, on an interior surface portion of the dose dial member.

The dose dial member may comprise a circumferential wall having an exterior peripheral surface which is accessible for user interaction and an interior peripheral surface. The first serrated transversal surface may be arranged along at least a portion of the interior peripheral surface which will maximise the diameter on which the serrated surfaces serving to hold the torque of the drive spring during dose setting interact, thereby enabling the use of a softer bias structure, such as a low rate spring, in the axial ratchet mechanism.

The second serrated transversal surface may form part of a spring retention member adapted to hold a first end portion of the drive spring. Such a spring retention member may be provided in the form of a spring base being arranged axially and rotationally fixed in the housing. Realising the second serrated transversal surface as part of the spring retention member also eliminates the need for an additional dedicated ratchet component in the device since a spring retention member has to be present anyway.

The drive member may be adapted to hold a second end portion of the drive spring, whereby the drive spring is torsionally strained when the activation button is in the inactivated position and the dose dial member is rotated to set a dose, and released to rotate the drive member when the activation button is moved to the activated position. The drive spring may e.g. be a helical spring or a spiral spring.

In particular embodiments of the invention the dose dial member comprises a toothed interior collar and the drive member comprises an exterior surface portion having axially extending teeth, e.g. arranged in circumferential distribution. The axially extending teeth are configured to engage with the toothed collar when the drive member is in the dose setting position whereby the dose dial member and the drive member are rotationally interlocked. The dimension of the axially extending teeth is chosen such that during distal movement of the drive member from the dose setting position to the dose expelling position the axially extending teeth move axially out of alignment with the toothed interior collar, whereby the drive member is rotationally disengaged from the dose dial member.

The means for receiving a drug reservoir may e.g. comprise a reservoir holding structure, such as e.g. a cartridge holder, attached, or adapted for attachment, to the housing, or a coupling structure in the form of a material or geometry in or on the housing adapted to secure a desired position of the drug reservoir, or of the reservoir holding structure, relative to the housing, and a received reservoir is, naturally, a reservoir which is attached to the housing by way of such means.

As used herein, the terms “distal” and “proximal” denote positions at or directions along the drug delivery device, where “distal” refers to the drug outlet end of the drug delivery device and “proximal” refers to the end opposite the drug outlet end.

Further, to avoid any doubt, it is noted that the serrated transversal surfaces are transversal with respect to the longitudinal axis of the housing. The transversal surfaces may be at right angles to the longitudinal axis, or alternatively, in some embodiments, at respective acute and obtuse angles to the longitudinal axis.

In the present specification, reference to a certain aspect or a certain embodiment (e.g. “an aspect”, “a first aspect”, “one embodiment”, “an exemplary embodiment”, or the like) signifies that a particular feature, structure, or characteristic described in connection with the respective aspect or embodiment is included in, or inherent of, at least that one aspect or embodiment of the invention, but not necessarily in/of all aspects or embodiments of the invention. It is emphasized, however, that any combination of the various features, structures and/or characteristics described in relation to the invention is encompassed by the invention unless expressly stated herein or clearly contradicted by context.

The use of any and all examples, or exemplary language (e.g., such as, etc.), in the text is intended to merely illuminate the invention and does not pose a limitation on the scope of the same, unless otherwise claimed. Further, no language or wording in the specification should be construed as indicating any non-claimed element as essential to the practice of the invention.

### BRIEF DESCRIPTION OF THE DRAWINGS

In the following the invention will be further described with references to the drawings, wherein

FIG. 1 is a cross-sectional view of a proximal portion of a drug delivery device according to an exemplary embodiment of the invention,

FIG. 2 is a perspective view showing a proximal ratchet part in the drug delivery device, and

FIG. 3 is a perspective view showing a distal, mating ratchet part in the drug delivery device.

In the figures like structures are mainly identified by like reference numerals.

### DESCRIPTION OF EXEMPLARY EMBODIMENTS

When in the following relative expressions, such as “upper” and “lower”, and “clockwise” and “counter-clockwise”, are used, these refer to the appended figures and not necessarily to an actual situation of use. The shown figures are schematic representations for which reason the configuration of the different structures as well as their relative dimensions are intended to serve illustrative purposes only.

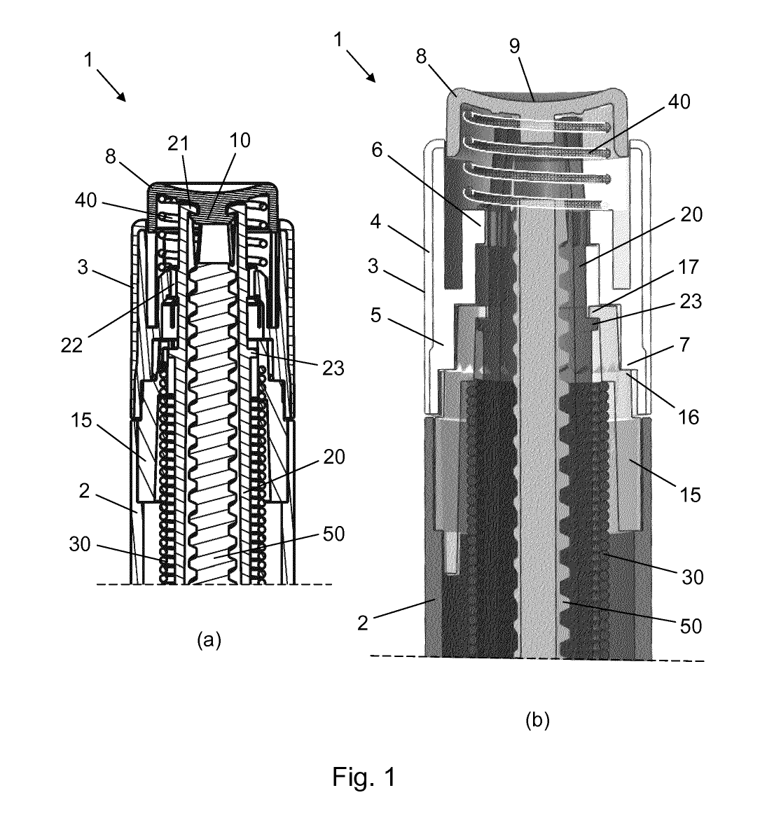

FIG. 1 shows a proximal portion of a drug injection device 1 according to an exemplary embodiment of the invention. While FIG. 1(a) is a conventional longitudinal section view FIG. 1(b) is an alternative longitudinal section view which is included for the sake of clarity. The remaining portion of the injection device 1 is left out because of its irrelevance to the understanding of the present invention. However, it is noted that of a plurality of possible ways to construct said portion one exemplary is described and shown in the aforementioned WO 2014/161952, of which page 11, line 29-page 12, line 24, and page 15, line 5-8, are hereby specifically incorporated by reference.

The injection device 1 comprises a housing 2 adapted to accommodate a dose setting mechanism for setting of a dose to be delivered from an attached drug cartridge (not shown) as well as a dose expelling mechanism for delivery of the set dose. The housing 2 is a tubular shell which at its proximal end is connected with a dose dial 3. The dose dial 3 is rotatable about a longitudinal centre axis of the housing 2 and comprises an exterior portion 4 adapted for operation by a user and an interior portion 5. At the proximal end of the dose dial 3 an injection button 8 is arranged. The injection button 8, which has a push face 9 for reception of a user's index finger or thumb, is axially movable between an extended position (as shown in FIG. 1), defining a dose setting mode of the injection device 1, and a depressed position, defining a dose delivery mode of the injection device 1.

The injection button 8 is biased towards the extended position by a button spring 40, which is a helical compression spring seated between an interior surface portion of the injection button 8 and a toothed collar 6 in the interior portion 5 of the dose dial 3. An inwardly protruding harpoon member 10 (only visible in FIG. 1(a)) engages a catch portion 21 of a drive tube 20 and serves to axially interlock the injection button 8 and the drive tube 20, thereby making the drive tube 20 axially movable between a proximal dose setting position (corresponding to the injection button 8 being in the extended position) and a distal dose expelling position (corresponding to the injection button 8 being in the depressed position).

The drive tube 20 extends longitudinally within the housing 2 and surrounds a centrally located threaded piston rod 50. The piston rod 50 is in threaded engagement with a nut member (not shown) in the housing 2 and is adapted to engage and advance a piston (not shown) in the drug cartridge for expelling of a set dose by rotation relative to the housing 2. The rotation of the piston rod 50 is caused partly by the drive tube 20, as will be clear from the below.

A spring base 15, being axially and rotationally fixed with respect to the housing 2, serves to hold a proximal end portion of a drive spring 30. The drive spring 30 is a helical torsion spring and a distal end portion thereof is rotationally fixed to a distal portion of the drive tube 20. The drive tube 20 has an exterior circumferential toothing 22 which is in rotational engagement with the toothed collar 6 when the injection button 8 is in the extended position, thereby rotationally interlocking the drive tube 20 and the dose dial 3 in the dose setting mode.

The button spring 40, which acts between the injection button 8 and the toothed collar 6, tends to press the injection button 8 out of the housing 2. The extent to which the injection button 8 can move out of the housing 2 is defined by a transversal flange 23 on the drive tube 20 which abuts diametrically opposed inward protrusions 17 at the proximal end portion of the spring base 15, limiting the axial proximal movement of the drive tube 20 and thereby of the catch portion 21.

A stepped portion of the spring base 15 has a serrated rim 16 which is capable of slipping engagement with a mating serrated rim 7 of the interior portion 5 during a user's rotation of the dose dial 3. The serrated rim 16 and the serrated rim 7 are configured to allow such slipping engagement during both clockwise and counter-clockwise rotation of the dose dial 3 relative to the housing 2.

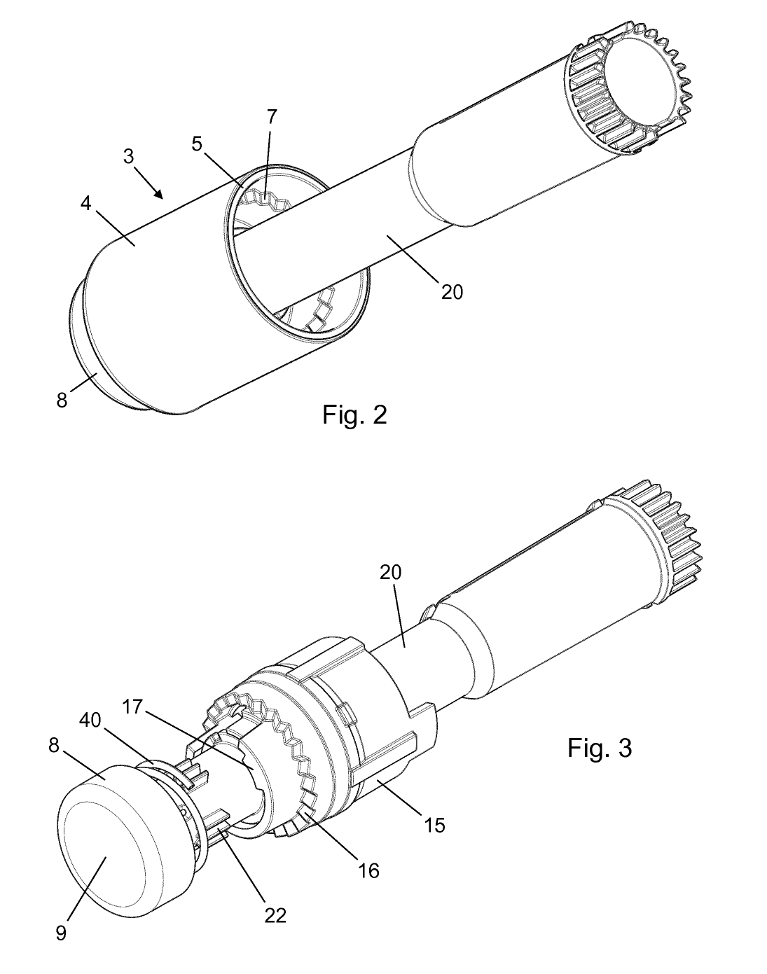

FIG. 2 is a perspective distal view of selected components of the injection device 1, more specifically the dose dial 3, the injection button 8, and the drive tube 20. The figure particularly shows the construction of the dose dial 3 with the exterior portion 4, the interior portion 5, and the serrated rim 7. The dose dial 3 can either be a unitary structure or can consist of a plurality of sub-components being axially and rotationally interlocked.

FIG. 3 is a perspective proximal view of selected components of the injection device 1, more specifically the injection button 8, the button spring 40, the spring base 15, and the drive tube 20. The figure particularly shows the construction of the spring base 15 with the inward protrusions 17 (only one is visible) and the serrated rim 16. The serrated rim 7 and the serrated rim 16 establish an axial ratchet connection between the dose dial 3 and the spring base 15. Said ratchet connection is dimensioned to hold the torque generated by the drive spring 30 during dose setting, with the support of the button spring 40.

To set a dose to be administered from the injection device 1 the user applies a torque to the dose dial 3 and turns the dose dial 3 relative to the housing 2 about the longitudinal centre axis. The serrated interface between the interior portion 5 and the spring base 15 causes the dose dial 3 to reciprocate slightly with respect to the housing 2 as respective serrations on the serrated rim 7 slide along respective serrations on the serrated rim 16 to disengage the two surfaces and as the respective serrations on the serrated rim 7 slide back along the following serrations on the serrated rim 16 to the bring the surfaces back into engagement. The proximal movement of the dose dial 3 which disengages the serrated rim 7 from the serrated rim 16 is performed against the bias force of the button spring 40, as it is being compressed between the injection button 8 and the toothed collar 6, while the distal movement of the dose dial which restores engagement between the serrated rim 7 and the serrated rim 16 is caused by the button spring 40, expanding in response to the respective summits being surpassed.

As the dose dial 3 changes angular position relative to the housing 2 the drive tube 20, being rotationally locked to the dose dial 3 via the interface between the toothing 22 and the toothed collar 6, changes angular position relative to the spring base 15, whereby the drive spring 30 is strained torsionally. The angular displacement of the drive tube 20 is directly reflected in a helical displacement, along the longitudinal centre axis, of a scale drum (not shown) carrying a plurality of dose related numerals, at least one of which is visible to the user through a window (not visible) in the housing 2 to indicate the size of the presently set dose.

During and following the setting of a dose when the user releases her grip on the dose dial 3 the strained drive spring 30 is prevented from unwinding by the engagement between the serrated rim 7 and the serrated rim 16 enforced by the button spring 40, and the dose dial 3 thus remains in the chosen position relative to the housing 2. To administer the set dose the injection button 8 is depressed against the housing 2, whereby the drive tube 20 is displaced distally into rotational locking engagement with a piston rod driving member (not shown), and the toothing 22 disengages from the toothed collar 6 to release the drive spring 30. As the drive spring 30 unwinds the drive tube 20 rotates together with the piston rod driving member, whereby the piston rod 50 rotates and moves forward, in the distal direction, due to its threaded engagement with the nut member in the housing 2. The distance by which the piston rod 50 is thus moved is defined by the distance which the scale drum is allowed to move in the housing from the initial dose set position to an end-of-dose position.

The arrangement of the button spring 40 between the injection button 8 and the toothed collar 6 serves a dual purpose in that not only does the button spring 40 provide the necessary axial bias to the interior portion 5 to prevent the serrated rim 7 from unintentionally slipping over the serrated rim 16, at the same time it also provides the necessary axial bias to the injection button 8 to return the injection button 8 to the extended position and the drive tube 20 to the proximal dose setting position following a completed dose delivery (and discontinuation of the user's depressive force), thereby automatically placing the injection device 1 in the dose setting mode ready for the preparation of a new injection. An automatic return of the injection button is a common feature in automatic injection devices and the present solution cost-efficiently eliminates the need for further components by utilising the same means that causes this return to also both provide the necessary bias to the ratchet connection in the dose setting mechanism and cause the re-coupling of the drive tube 20 with the dose dial 3. In view of the popularity of prefilled injection devices, which are discarded after use, this solution also entails significantly less wastage.

It is noted that the respective serrations on the serrated rim 7 and the serrated rim 16 may be asymmetrical in configuration to compensate for the fact that the drive spring 30 provides a positive contribution to a dose decreasing rotation of the dose dial 3. The efforts required of the user to increase, respectively decrease the dose can thus be designed to be comparable.

Further, the serrated rim 7 and the serrated rim 16 may be configured such that the angular displacement of the dose dial 3 relative to the housing 2 required to cause the serrated rim 7 to displace angularly by a single serration relative to the serrated rim 16 corresponds to a single unit increase or decrease of the dose. The tactile and audible output provided by the ratchet connection during the riding of the serrated rim 7 over the serrated rim 16 thus allows users with e.g. impaired vision to determine the set dose by counting the clicks from, and/or the jerks of, the dose dial 3.

1. A drug delivery device comprising:
a housing extending along a longitudinal axis,
a dose dial member being rotatable relative to the housing to set a dose of drug to be expelled from an attached reservoir and comprising a first serrated transversal surface,
a second serrated transversal surface being axially and rotationally fixed with respect to the housing, and being configured for slipping engagement with the first serrated transversal surface in a bi-directional ratchet interface in response to the dose dial being rotated relative to the housing to increase or decrease the dose, and
a dose delivery mechanism for expelling a set dose, the dose delivery mechanism comprising
a piston rod,
a torsion spring for providing energy to drive the piston rod,
an activation button being axially movable relative to the housing between an inactivated position in which the torsion spring is retained and an activated position in which the torsion spring is released to cause an expelling of the set dose, the activation button being biased towards the inactivated position by a bias structure arranged to act between the dose dial member and the activation button, the bias structure thereby further biasing the first serrated transversal surface towards the second serrated transversal surface, and
a drive member being rotationally biased by the torsion spring,
wherein the drive member is axially fixed to the activation button and axially movable relative to the housing between a dose setting position in which the drive member is rotationally fixed to the dose dial member and a dose expelling position in which the drive member is rotationally freed from the dose dial member and rotationally interlocked with the piston rod.

2. A drug delivery device according to claim 1, wherein the first serrated transversal surface is formed on an interior surface of the dose dial member.

3. A drug delivery device according to claim 1, wherein the first serrated transversal surface is arranged along an internal periphery of the dose dial member.

4. A drug delivery device according to claim 1, wherein the second serrated transversal surface forms part of a spring retention member adapted to hold an end portion of the drive spring.

5. A drug delivery device according to claim 1, wherein the bias structure is a unitary element comprising a distal end portion abutting a proximally oriented portion of the dose dial member and a proximal end portion abutting a distally oriented portion of the activation button.

6. A drug delivery device according to claim 1, wherein the bias structure comprises a metallic compression spring.

7. A drug delivery device according to claim 1, wherein the dose dial comprises a toothed inner collar, and the drive member comprises an exterior surface portion having axially extending teeth,
wherein when the drive member is in the dose setting position the axially extending teeth engage with the toothed inner collar, and
wherein during movement of the drive member from the dose setting position to the dose expelling position the axially extending teeth disengage from the toothed inner collar.

8. A drug delivery device according to claim 1, wherein the activation button is arranged at a proximal end portion of the housing.
